(19) United States
(12) Patent Application Publication
(10) Pub. No.: US 2015/0359565 A1
(43) Pub. Date: Dec. 17, 2015

(54) TROCAR

(71) Applicant: OLYMPUS CORPORATION, Tokyo (JP)

(72) Inventor: Akira MATSUI, Tokyo (JP)

(73) Assignee: OLYMPUS CORPORATION, Tokyo (JP)

(21) Appl. No.: 14/835,860

(22) Filed: Aug. 26, 2015

Related U.S. Application Data
(63) Continuation of application No. PCT/JP2014/056339, filed on Mar. 11, 2014.

(30) Foreign Application Priority Data
Aug. 22, 2013 (JP) 2013-172474

(51) Int. Cl. A61B 17/34

(52) U.S. Cl. CPC A61B 17/3476; A61B 17/3421; A61B 17/3474; A61B 18/1482

(57) ABSTRACT

A trocar comprising: a housing; an insertion tube that is integral with the housing and is inserted into a subject; a power transmission coil that is disposed inside of the housing and generates AC magnetic field to be applied to an insertion hole into which a treatment tool is to be inserted; a radiator that transmits heat generated from the power transmission coil; and a heat insulator disposed on a side of the subject between the radiator and the subject, the heat insulator having lower thermal conductivity than the radiator.

### CROSS REFERENCE TO RELATED APPLICATIONS

This application is a continuation of PCT International Application No. PCT/JP2014/056339, filed on Mar. 11, 2014, the entire content of which is incorporated by this reference, and claims priority to Japanese Patent Application No. JP 2013-172474, filed on Aug. 22, 2013, the entire content of which is incorporated by this reference.

### BACKGROUND

1. Technical Field

The present invention relates to a trocar to feed power to a treatment tool wirelessly.

2. Background Art

A trocar is integrally combined with an inner needle having a sharp puncture needle at the forward end, and the inner needle in such a state is punctured through a body wall of a patient so as to be inserted into the abdominal cavity. After being inserted into the abdominal cavity, the inner needle is removed so as to leave the trocar at the body wall, and then the trocar is used as a guide tube for a treatment tool that is for treatment in the abdominal cavity.

Some treatment tools inserted into a trocar are connected to a cable to receive power required for the treatment. Such a cable hinders the manipulation by an operator during operation and degrades the operability.

To solve this problem, JP H11-128242 A discloses a technique of supplying power from a power-transmission coil of a trocar to a power-reception coil of a treatment tool that is inserted into the trocar.

An energy treatment tool for surgical operation, such as a diathermy knife or an ultrasonic knife, however, requires relatively large power of about 10 W to 100 W for treatment, and so the power-transmission coil may generate heat and temperature of the trocar housing in contact with the patient may rise.

An embodiment of the present invention provides a trocar capable of suppressing temperature rise at the housing.

### BRIEF DESCRIPTION OF THE DRAWINGS

FIG. 1 schematically illustrates the usage state of an operating system including a trocar of a first embodiment.

FIG. 2 illustrates a circuit configuration of the operating system including the trocar of the first embodiment.

FIG. 3 is a partial cross sectional view of the trocar of the first embodiment.

FIG. 4 is a perspective view of a radiator and a heat insulator of the trocar of the first embodiment.

FIG. 5A is a cross sectional view of a radiator and a power transmission coil of a trocar of the first embodiment.

FIG. 5B is a cross sectional view of a radiator and a power transmission coil of a trocar of the first embodiment.

FIG. 5C is a cross sectional view of a radiator and a power transmission coil of a trocar of the first embodiment.

FIG. 6A is a perspective view of a radiator as a modification example of the first embodiment.

FIG. 6B is a perspective view of a radiator as another modification example of the first embodiment.

FIG. 7 is an exploded view of a radiator as a modification example of the first embodiment.

FIG. 8 schematically illustrates the usage state of an operating system including a trocar of a second embodiment.

FIG. 9 illustrates a circuit configuration of the operating system including the trocar of the second embodiment.

FIG. 10 illustrates the configuration to describe a flow channel for gas in the operating system including the trocar of the second embodiment.

### MODE FOR CARRYING OUT THE INVENTION

### First Embodiment

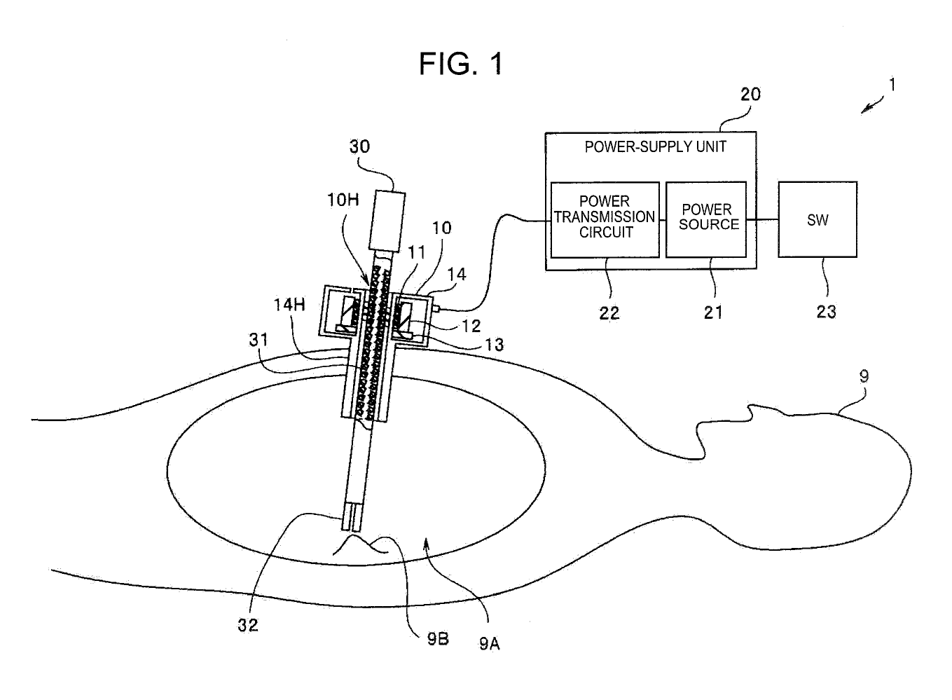

Referring firstly to FIGS. 1 to 4, the following describes an operating system 1 including a trocar 10 of a first embodiment. As illustrated in FIG. 1, the operating system 1 comprises the trocar 10, a power-supply unit 20, a treatment tool 30, and a switch 23. Although the operating system 1 allows an endoscope or the like also to be inserted into an abdominal cavity 9A of a subject 9 via another trocar, the descriptions thereof are omitted.

The trocar 10 of the present embodiment comprises a housing 14, an insertion tube 14H, a power transmission coil 11, a radiator 12, and a heat insulator 13. The long and thin insertion tube 14H that can be extended from a lower part of the housing 14 can be inserted into the subject 9. The housing 14 and the insertion tube 14H may be made of the same material and may be configured integrally. The housing 14 has an insertion hole 10H at the center, through which the treatment tool 30 can be to be inserted. The insertion hole 10H can be a through hole that can be extended to the forward end of the insertion tube 14H.

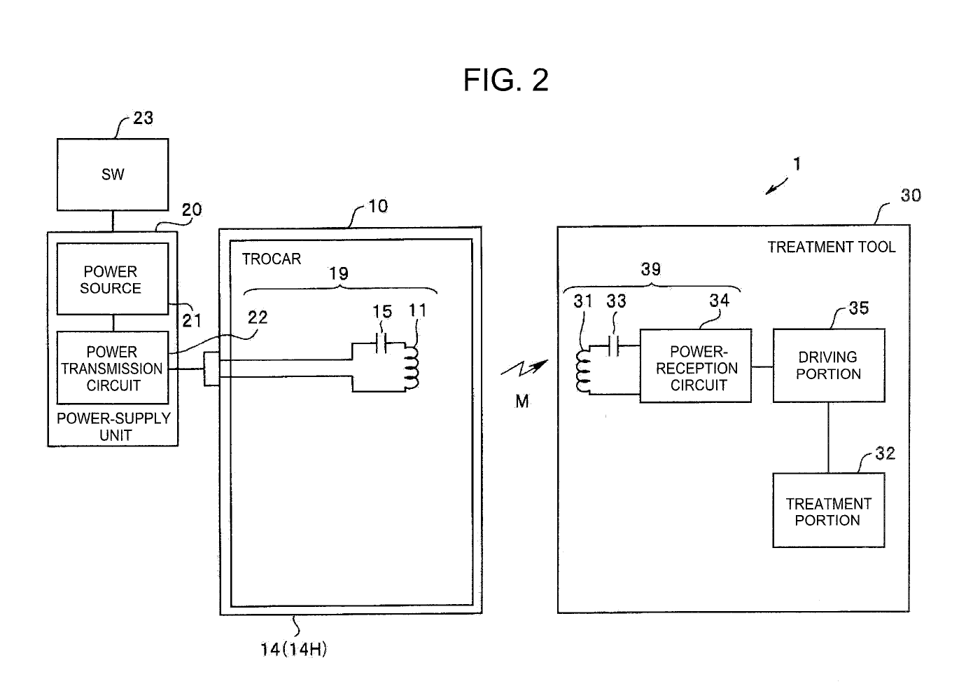

The treatment tool 30 can be inserted into the abdominal cavity 9A through the insertion hole 10H. The treatment tool 30 of the present embodiment can be a bipolar electrosurgical knife for treatment, such as incision and coagulation by applying high-frequency electric energy to a treated part 9B such as blood vessel that can be pinched with a treatment portion 32. The treatment tool 30 provides treatment using power that can be wirelessly received at a power-reception portion 39 (see FIG. 2), and so no cable is connected thereto for power supplying, meaning good operability.

The power-supply unit 20 to output AC power to the power transmission coil 11 of the trocar 10 comprises a power source 21 and a power transmission circuit 22. The power source 21 outputs high-frequency and large power of about 10 W to 100 W, for example. As illustrated in FIG. 2, a power transmission portion 19 that generates AC magnetic field from the power supplied from the power source 21 comprises the power transmission coil 11, a power-transmission capacitor 15 and a power transmission circuit 22. The power transmission coil 11 can be connected in series with the power-transmission capacitor 15, which make up a power-transmission side LC serial resonance circuit that generates AC magnetic field at a predetermined resonance frequency FR1. The power source 21 outputs AC power of the resonance frequency FR1. Instead of the power-transmission capacitor 15, floating capacity of the power transmission coil 11 may be used in another configuration. The power transmission circuit 22 comprises an impedance matching circuit (not illustrated) for impedance matching with the power source 21 and the resonance circuit.

FIG. 1 and FIG. 2 illustrate the power-transmission capacitor 15 that can be disposed in the trocar 10 and the power transmission circuit 22 that can be disposed in the power-supply unit 20. Instead, the power-transmission capacitor 15 and the power transmission circuit 22 may be disposed in the trocar 10 or in the power-supply unit 20.

The switch 23 connected to the power-supply unit 20 may be a foot switch for ON/OFF control of the power output from the power-supply unit 20.

As described above, the treatment tool 30 has the power-reception portion 39 including a power-reception coil that can be inductively coupled with the power transmission coil 11 of the power transmission portion 19 and receives power wirelessly via the AC magnetic field. As illustrated in FIG. 2, the power-reception portion 39 comprises the power-reception coil 31, a power-reception capacitor 33 and a power-reception circuit 34. The power-reception coil 31 can be connected in series with the power-reception capacitor 33, which make up a power-reception side LC serial resonance circuit that effectively receives AC magnetic field at a predetermined resonance frequency FR2. The resonance frequency FR2 of the power-reception side LC serial resonance circuit is substantially the same as the resonance frequency FR1 of the power-transmission side LC serial resonance circuit, and the operating system 1 performs wireless power exchange effectively based on a magnetic resonance phenomenon. The resonance frequencies FR1 and FR2 may be selected appropriately in the range of about 100 kHz to 20 MHz, from which a frequency that can be allowed for use by law, e.g., 13.56 MHz is preferably selected.

Instead of the power-reception capacitor 33, floating capacity of the power-reception coil 31 may be used in another configuration. The power-reception circuit 34 rectifies an AC signal that the power-reception coil 31 receives into a DC signal, followed by smoothing, and then regulates it with a DC/DC converter to have voltage to be supplied to a driving portion 35. The power-reception circuit 34 comprises an impedance matching circuit (not illustrated) for impedance matching with the driving portion 35 and the resonance circuit. The driving portion 35 converts power from the power-reception circuit 34 into power suitable for driving at the treatment portion 32. The treatment portion 32 (such as an electrosurgical knife) receives a driving signal at the frequency of 350 kHz and at the voltage of about hundreds Vpp from the driving portion 35, for example.

The power-reception coil 31 that can be of a long and thin solenoid type can be disposed in the long and thin insertion portion of the treatment tool 30 to be inserted into the subject 9 along the longer axis direction. The power-reception coil 31 that receives AC magnetic field generated from the power transmission coil 11 of the trocar 10 has a center axis that substantially agrees with the center axis of the insertion portion of the treatment tool 30. The power-reception coil 31 may have a length of about 100 mm or more and 200 mm or less, for example, so as to allow a part thereof to be inserted always into the power transmission coil 11, and also the power-reception coil 31 may have a length that can be disposed over the total length of the insertion portion of the treatment tool 30. The power-reception coil 31 may be surrounded with insulating resin at the periphery, for example.

On the other hand, the power transmission coil 11 that can be disposed inside of the housing 14 so as to be wound around the insertion hole 10H of the trocar 10 receives AC power and when generating AC magnetic field, generates heat due to Joule heat.

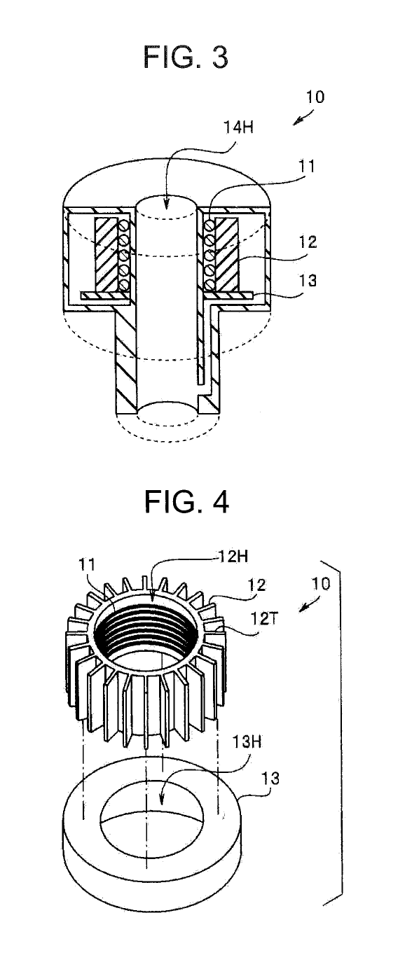

The radiator 12 that can be a cylindrical member having a hollow body radiates heat generated from the power transmission coil 11. This can prevent excessive temperature rise at the power transmission coil 11. The radiator 12 serves as a heat sink to which heat generated from the power transmission coil 11 can be transmitted increases in temperature. As illustrated in FIG. 3 and FIG. 4, for example, the heat insulator 13 can be disposed under the radiator 12, i.e., on the side closer to the subject than the radiator 12, the heat insulator having lower thermal conductivity than the radiator 12. The heat insulator 13 can be a cylindrical member that surrounds the lower face of the radiator 12 and has a hollow inside. That is, the radiator 12 and the heat insulator 13 have a through hole 12H and a through hole 13H that are substantially the same in diameter, which make up an insertion opening of the trocar 10.

Even when the radiator 12 is heated, the heat insulator 13 can be disposed under the radiator 12, and so the temperature of the housing 14 on the lower side does not increase.

The radiator 12 preferably has thermal conductivity λ of 15 W/(m·K) or more. That is, the radiator 12 preferably is made of copper (λ=398 W/(m·K)), silicon (λ=168 W/(m·K)), aluminum nitride (λ=150 W/(m·K)), iron (λ=84 W/(m·K)), alumina (λ=32 W/(m·K)), silicon nitride: Si₃N₄ (λ=27 W/(m·K)) or stainless steel (λ=17 W/(m·K)), for example.

In order to prevent loss due to eddy current, the radiator 12 is preferably non-electrically conductive, and can be made of a ceramic material, such as aluminum nitride particularly preferably.

If the radiator 12 is made of an electrically-conductive material, the power transmission coil 11 used has to comprise a core (made of copper or the like) that is surrounded with an insulating material. On the other hand, if the radiator 12 is made of a non-electrically conductive material, the power transmission coil may comprise a core without being surrounded with an insulating material.

The radiator 12 illustrated in FIG. 4, for example, has cooling fins 12T for cooling efficiency by increasing the surface area. The cooling fins 12T may have a rod shape, for example, as long as it does not hinder the current of air between fins. Warmed air moves naturally from down to up in the vicinity of the radiator 12 so as to generate convection of air in the housing, whereby heat at the radiator 12 can be discharged to the outside. The housing 14 of the trocar 10 may have openings (not illustrated) for ventilation at the upper face and the side face. Air flowing from the opening at the side face into the housing can be warmed by the radiator 12 and can be discharged from the opening at the upper face.

Meanwhile, the heat insulator 13 preferably has thermal conductivity λ of 0.3 W/(m·K) or less, and is 0.1 W/(m·K) or less particularly preferably. That is, the heat insulator 13 preferably can be made of resin material, such as epoxy resin (λ=0.21 W/(m·K)), silicone resin (λ=0.16 W/(m·K)), urethane resin (λ=0.034 W/(m·K)), silicon sponge (λ=0.08 W/(m·K)) or urethane foam (λ=0.029 W/(m·K)) made of such resin in a foamed shape, or glass wool (λ=0.045 W/(m·K)), for example. A space may be formed, and air (λ=0.024 W/(m·K)) may be used as the heat insulator 13.

In an embodiment the radiator 12 of the trocar 10 does not increase in temperature excessively because of so-called natural cooling.

The power transmission coil 11 of the trocar 10 is not excessively heated due to the radiator 12 even when large power is applied to the power transmission coil 11. Since the heat insulator 13 can be disposed under the radiator 12 (the subject side), temperature rise of the housing can be suppressed, especially at a portion of the housing 14 that can be in contact with the subject 9.

### Modification Example

The following describes trocars 10A to 10F as modification examples of the trocar 10 of the first embodiment. All of the trocars 10A to 10F of the modification examples have the advantageous effects of the trocar 10 of the first embodiment, and have other additional effects.

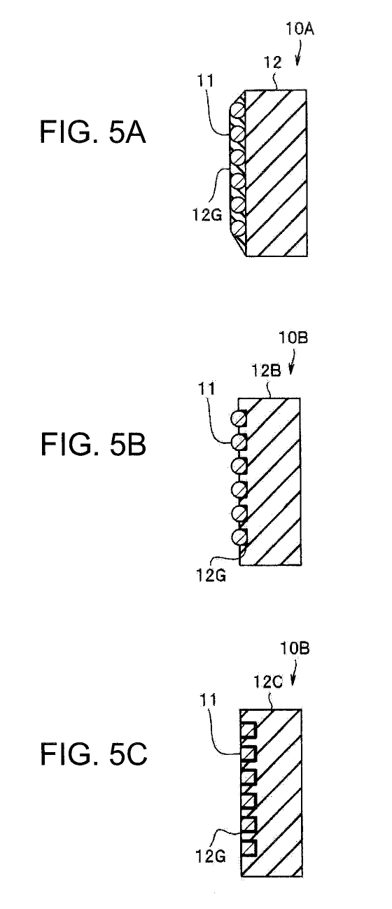

The trocar 10A as modification example 1 in FIG. 5A comprises a high thermal conductivity material 12G such as thermal grease or a sheet made of a high thermal conductivity material so as to cover the power transmission coil 11. The thermal grease may contain silicone resin as a major component, to which metal particles such as silver are mixed for better thermal conductivity. The trocar 10A has good heat transmission efficiency.

The trocar 10B as modification example 2 in FIG. 5B comprises a power transmission coil 11 disposed in a spiral-shaped groove at the inner periphery of a radiator 12B. The trocar 10B is configured to facilitate the disposition of the power transmission coil 11 in a predetermined shape at the inner periphery of the radiator 12.

The trocar 10C as modification example 3 in FIG. 5C comprises a power transmission coil 11 having a core that can be a rectangle in cross section, and the core can be inserted into a rectangular groove of a radiator 12C. The trocar 10C enables more effective transmission of heat generated at the power transmission coil 11 to the radiator 12. The core of the power transmission coil 11 that can be a circle in cross section may be inserted into a semicircular groove. That is, the radiator 12 having a groove that can be fitted with the core of the power transmission coil 11 can widen the contact area between the power transmission coil 11 and the radiator 12 and so can improve heat transmission efficiency.

In the configuration of the core of the power transmission coil 11 inserted in the spiral-shaped groove of the radiator as well, the high thermal conductivity material 12G, e.g., thermal grease, is preferably disposed at a gap between the groove and the core.

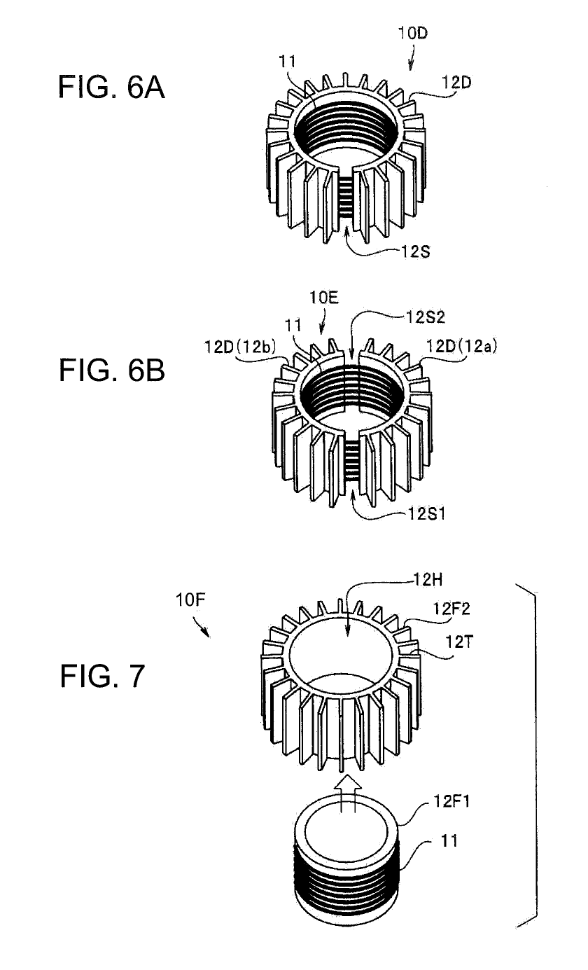

The trocar 10D as modification example 4 in FIG. 6A comprises a radiator 12D made of an electrically-conductive metal material, in which a slit 12S can be formed in the direction orthogonal to the circumferential direction. A metal material can be easily processed and can be less expensive as compared with a ceramic material. A radiator made of an electrically-conductive material, however, generates an eddy current flowing therein and generates loss when an AC magnetic field is applied. The radiator 12D having the slit 12S can reduce such loss from eddy current allowing that the radiator 12D has conductivity. The slit 12S may be air gap or may be made of a non-electrically conductive material.

The trocar 10E as modification example 5 in FIG. 6B comprises a radiator 12E made up of a plurality of metal members (electrically-conductive material) 12a and 12b that are electrically insulated by a plurality of slits 12S1 and 12S2.

The trocar 10F as modification example 5 in FIG. 7 comprises a radiator 12F, including an inner tubular member 12F1 around which a power transmission coil 11 can be wound, and an outer tubular member 12F2 joined to the outer periphery of the inner tubular member 12F1. Winding of the power transmission coil 11 around the outer periphery of the inner tubular member 12F1 can be easier than winding at the inner periphery.

Further, since the power transmission coil 11 of the trocar 10F can be held between the inner tubular member 12F1 and the outer tubular member 12F2, heat generated at the power transmission coil 11 can be effectively transmitted to the radiator 12F. The inner tubular member 12F1 and the outer tubular member 12F2 may be made of the same material or different materials. At least one of the inner tubular member 12F1 and the outer tubular member 12F2 may be made of a high thermal conductivity material, and both of them are made of a high thermal conductivity material particularly preferably. Preferably the inner tubular member 12F1 and the outer tubular member 12F2 are in intimate contact via a high thermal conductivity material, such as thermal grease.

### Second Embodiment

The following describes an operating system 1G including a trocar 10G that is a second embodiment. Since the trocar 10G is similar to the trocar 10, the same reference numerals are assigned to common elements, and their descriptions are omitted.

While the radiator 12 in the trocar 10 can be naturally cooled, the trocar 10G can be forced-cooled. Then temperature rise at the housing of the trocar 10G can be suppressed as compared with the trocar 10, and the configuration described later has other additional advantageous effects.

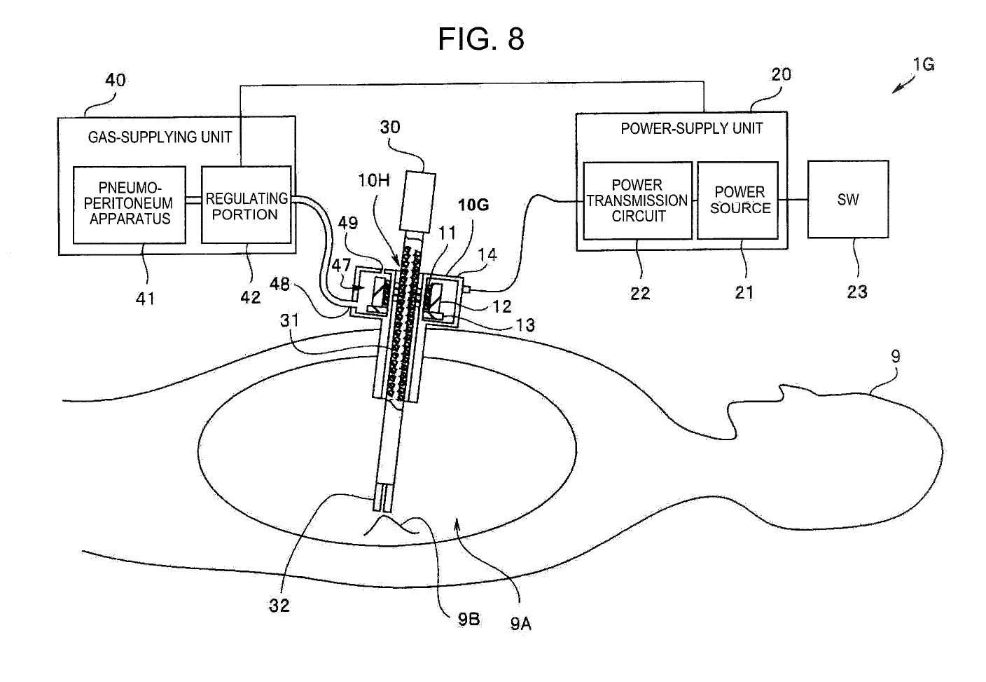

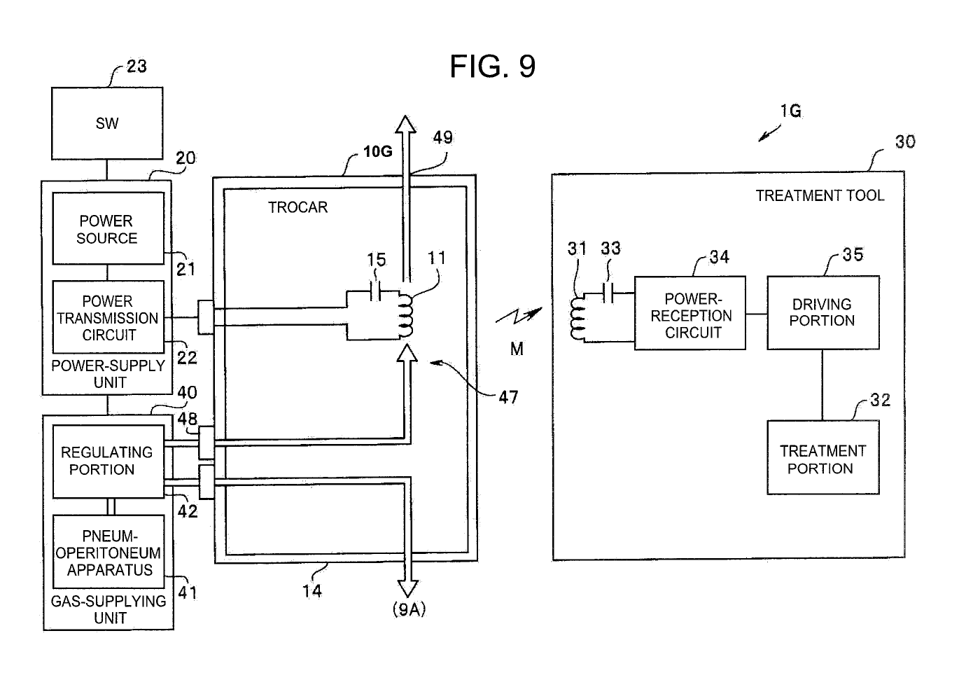

That is, as illustrated in FIG. 8 and FIG. 9, the operating system 1G comprises a gas-supplying unit 40 to supply gas for forced-cooling of the radiator 12, in addition to the elements of the operating system 1.

A pneumoperitoneum apparatus 41 of the gas-supplying unit 40 fills an abdominal cavity to be treated with gas such as carbon dioxide to expand the abdominal cavity, thus keeping the viewing field for observation required to the treatment of the abdominal cavity and so allowing the operator to understand the treatment process sufficiently. Filling of gas into the abdominal cavity by the pneumoperitoneum apparatus is performed under the control of pressure of gas from a gas supplying source (not illustrated) such as a gas cylinder using a pressure-reducing device or a valve so as to keep the pressure in the abdominal cavity at the set pressure.

The trocar 10G comprises an inlet 48 through which gas to cool the radiator 12 can be introduced, a flow channel 47 including a pipe through which the gas flows, and an outlet 49 through which the gas can be discharged. The inlet 48 can be disposed at a lower part of the side face of the housing 14, and the outlet 49 can be disposed at an upper part of the side face or at the upper face.

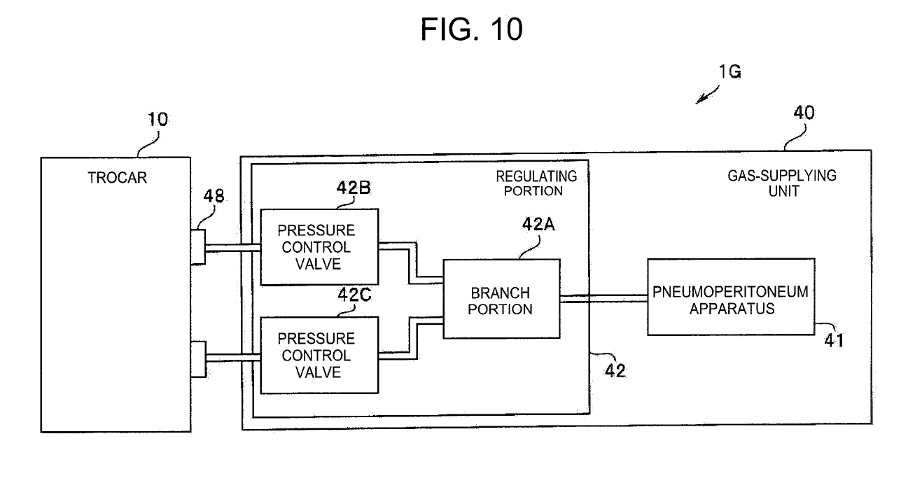

The gas-supplying unit 40 comprises a regulating portion 42 that divides gas supplied from the pneumoperitoneum apparatus 41 into a path to flow in the abdominal cavity and a path to flow in the flow channel 47 to cool the radiator 12. For instance, as illustrated in FIG. 10, the regulating portion 42 comprises a branch portion 42A, a pressure control valve 42B that can be controlled based on the pressure inside of the abdominal cavity and a pressure control valve 42C to control the flow rate of gas to flow in the flow channel 47.

Each of the pressure control valves 42B and 42C may be a passive one that regulates pressure on the secondary side based on balance between an elastic member such as a spring disposed internally and the pressure in the flow channel, or an active one such as an electromagnetic valve. When the pressure control valve 42B is an active control valve, the pressure on the secondary side (abdominal cavity side) can be detected by a pressure sensor (not illustrated) such as a diaphragm sensor, and the valve can be controlled so that applied pressure has a predetermined value. The pressure control valve 42C may have a configuration similar to that of the pressure control valve 42B, and can be adjusted so that the pressure on the secondary side has a value required for flowing at the sufficient flow rate for coil cooling.

At least a part of the regulating portion 42, e.g., the pressure control valve 42C may be disposed at the trocar 10, and the pressure control valve 42B may be disposed at the pneumoperitoneum apparatus 41.

The flow rate of gas supplied to the flow channel may be set at a predetermined amount, and it is preferable that the regulating portion 42 makes a flow-rate control portion (not illustrated) regulate the flow rate or the like in accordance with the temperature or the amount of power of the power transmission coil 11 so as to prevent excessive supply of gas. Although the temperature of the power transmission coil 11 may be detected by a temperature sensor (not illustrated), it can be estimated from the amount of power applied to the power transmission coil 11 as well. That is, since the temperature and the amount of power of the power transmission coil 11 have a proportional relationship, regulation of the flow rate or the like based on the amount of power has the same meaning as the regulation of the flow rate or the like based on the temperature of the power transmission coil.

For instance, control can be performed so that gas can be supplied to the flow channel 47 only when the temperature or the amount of power of the power transmission coil 11 is a predetermined value or more. Alternatively the flow rate of gas to be supplied to the flow channel 47 may be controlled so as to be in proportion to the temperature or the amount of power of the power transmission coil 11.

The regulating portion 42, which regulates the pressure control valve 42C based on information on the temperature of the power transmission coil 11 or on the amount of power from the power-supply unit 20, can improve the efficiency of gas supplying and so can be economical.

In another configuration, gas subjected to regulation by the pressure control valve 42B may be supplied to the abdominal cavity via the flow channel 47. The operating system configured to cool the power transmission coil 11 with gas for pneumoperitoneum can be simpler than the configuration of the operating system 1G. That is, it does not have to comprise the branch portion 42A and the pressure control valve 42C.

An apparatus exclusively used to supply fluid to cool the radiator may be provided. In this case, the fluid used may be liquid such as water.

The present invention is not limited to the above-described embodiments, and can be changed and modified variously without changing the gist of the present invention.

1. A trocar for use with a treatment tool for treatment of a subject, the trocar comprising:
a housing having an insertion hole configured to receive the treatment tool;
an insertion tube that is integral with the housing and is configured to be inserted into the subject;
a power transmission coil that is disposed inside of the housing and is configured to generate an AC magnetic field to be applied to the treatment tool being inserted into the insertion hole; and
a heat radiator configured to radiate heat generated from the power transmission coil.

2. The trocar according to claim 1, further comprising:
a heat insulator located between the radiator and the subject,
wherein the heat insulator has a lower thermal conductivity than the radiator.

3. The trocar according to claim 1,
wherein the radiator has a slit therein in a direction orthogonal to a radial direction of the radiator
wherein the radiator is made of an electrically conductive material.

4. The trocar according to claim 3,
wherein the radiator further comprises a plurality of slits.

5. The trocar according to claim 3,
wherein the radiator further comprises an inner tubular member having an outer periphery on which the power transmission coil is wound.

6. The trocar according to claim 5,
wherein the radiator further comprises
an outer tubular member, wherein the inner tubular member is located inside the outer tubular member.

7. The trocar according to claim 1, wherein the radiator has on an inner surface thereof a spiral-shaped groove, in which the power transmission coil is inserted.

8. The trocar according to claim 1, further comprising:
an inlet connecting portion configured to introduce a fluid into the trocar;
a flow channel being configured to flow the fluid in order to cool the heat radiator; and
an outlet connected to the flow channel, the outlet being configured to discharge the fluid.

9. The trocar according to claim 8,
wherein the fluid is gas supplied from a pneumoperitoneum apparatus.

10. The trocar according to claim 9,
wherein the gas is supplied into an abdominal cavity of the subject via the flow channel.

11. The trocar according to claim 8,
wherein the fluid to be supplied to the flow channel is regulated in accordance with at least a temperature of the power transmission coil or an amount of power of the power transmission coil.

12. The trocar according to claim 11,
wherein, when the temperature or the amount of power is a predetermined value or more, the fluid is supplied to the flow channel.
